SEQUENCE LISTING (6 sequences)

SEQ ID NO: 1 - DNA, 131 bp, Homo sapiens
  misc_signal (55)..(58): Frameshift site.
tcgacggtctccctccactgctgtagtaacccgggtccggggcctcggtggtgctcctgatgcccctcacccacccctgaagatcccaggtgggcgagggaatagtcagagggatcacaatccttcagctg

SEQ ID NO: 2 - DNA, 128 bp, Homo sapiens
  misc_signal (55)..(58): Frameshift site.
tcgacgccccagctccagtgctgcaggcacattgttccagggcctctgtggtgctcctgatgcccctcacccactgtcgaagatccccggtgggcgagggggcggcagggatccttctctctcagctg

SEQ ID NO: 3 - DNA, 86 bp, Homo sapiens
  misc_signal (10)..(13): Frameshift site.
tcgacgtgctcctgatgcccctcacccacccctgaagatcccaggtgggcgagggaatagtcagagggatcacaatccttcagctg

SEQ ID NO: 4 - DNA, 83 bp, Homo sapiens
  misc_signal (10)..(13): Frameshift site.
tcgacgtgctcctgatgcccctcacccactgtcgaagatccccggtgggcgagggggcggcagggatccttctctctcagctg

SEQ ID NO: 5 - DNA, 68 bp, Homo sapiens
  misc_signal (55)..(58): Frameshift site.
tcgacggtctccctccactgctgtagtaacccgggtccggggcctcggtggtgctcctgatgcccctg

SEQ ID NO: 6 - DNA, 68 bp, Homo sapiens
  misc_signal (55)..(58): Frameshift site.
tcgacgccccagctccagtgctgcaggcacattgttccagggcctctgtggtgctcctgatgcccctg

(19) United States
(12) Patent Application Publication
(10) Pub. No.: US 2004/0137454 A1
(43) Pub. Date: Jul. 15, 2004

(54) Assay for drug-induced recoding

(76) Inventors: Michael T Howard, Salt Lake City, UT (US); Raymond F Gesteland, Salt Lake City, UT (US); John F Atkins, Salt Lake City, UT (US)

Correspondence Address: ALAN J. HOWARTH, P.O. BOX 1909, SANDY, UT, 84091-1909, US

(21) Appl. No.: 10/472,729

(22) PCT Filed: Mar. 22, 2002
(86) PCT No.: PCT/US02/08909

(51) Int. Cl. C12Q 1/68; C12N 15/63

(52) U.S. Cl. 435/6; 435/320.1

(57) ABSTRACT

A tissue culture assay for measuring drug-induced recoding in regulating cellular polyamine levels is described. A DNA construct containing the renilla luciferase gene separated by sort cloning site from the firefly luciferase gene, both under the control of a single upstream SV40 promoter is provided. The cloning site contains the portion of antizyme gene known to contain the mRNA signals for polyamine stimulated frameshifting with the downstream firefly gene in the +1 position relative to the upstream renilla gene. A control construct is also produced with the genes in the same reading frame. Frameshifting efficiencies can be determined by comparing the ratio or firefly to renilla luciferase activity in parallel cell cultures.

### CROSS-REFERENCE TO RELATED APPLICATIONS

This application claims the benefit of U.S. Provisional Application No. 60/277,803, filed Mar. 22, 2001, which is hereby incorporated by reference in its entirety.

### BACKGROUND OF THE INVENTION

The present invention relates generally to assays for determining recoding in drug-induced regulation of genes, and, more particularly but not exclusively, to measuring drug-induced recoding in genes involved in regulating cellular polyamine levels.

“Recoding” has been defined as a phenomenon where the rules for translation decoding are temporarily altered through specific sites and signals built into the mRNA sequence. I. Brierly, Ribosomal Frameshifting on Viral RNAs, 76 J. Gen. Virol. 1885-1892 (1995); R. F. Gesteland & J. Atkins, Recoding: Dynamic Reprogramming of Translation, 65 Annu. Rev. Biochem 741-768 (1996). In some cases of recoding, special signals are far distant 3′ on the mRNA, M. J. Berry et al., Functional Characterization of the Eukaryotic SECIS Elements which Direct Seleno-cysteine Insertion at UGA Codons, 12 EMBO J. 2215-3322 (1993); W. A. Miller et al., New Punctuation for the Genetic Code: Luteovirus Gene Expression, 8 Sem. Virol. 3-13 (1997), but in the great majority of cases of recoding the signals are close to the recoding site.

In mammalian cells, three kinds of recoding have been described. First, redefinition of stop codons to sense codons (i.e., readthrough) allows synthesis of selenocysteine-containing proteins, A. Bock et al., Selenoprotein Synthesis: An Expansion of the Genetic Code, 16 Trends BiochenL Sci. 463-467 (1991); S. C. Low & M J. Berry, Knowing When Not to Stop: Selenocysteine Incorporation in Eukaryotes 21 Trends Biochem Sci. 203-208 (1996), and synthesis of elongated proteins in many RNA viruses, such as Moloney murine leukemia virus (MuLV), Y. Yoshinaka et al., Murine Leukemia Virus Protease Is Encoded by the Gag-Pol Gene and Is Synthesized through Suppression of an Amber Termination Codon, 82 Proc. Nat'l Acad. Sci. USA 1618-1622 (1985). Second, +1 frameshifting regulates expression of ornithine decarboxylase antizyme. The system is autoregulatory and depends on the concentration of polyamines. S. Hayashi et al., Ornithine Decarboxylase Antizyme: A Novel Type of Regulatory Protein, 21 Trends Biochem. Sci. 27-30 (1996). Third, −1 frameshifting is used to synthesize the GagPol precursor polyprotein in retroviruses that have gag, (pro), and pol genes in different reading frames (except spumaretroviruses, J. Enssle et al., Foamy Virus Reverse Transcriptase Is Expressed Independently from the Gag Protein, 93 Proc. Nat'l Acad. Sci. USA 4137-4141 (1996)). Examples are the mouse mammary tumor virus (MMTV) gag-pro frameshift, T. Jacks et al., Two Efficient Ribosomal Frameshifting Events Are Required for Synthesis of Mouse Mammary Tumor Virus Gag-related Polyproteins, 84 Proc. Nat'l Acad. Sci. USA 4298-4302 (1987); R. Moore et al., Complete Nucleotide Sequence of a Milk-transmitted Mouse Mammary Tumor Virus: Two Frameshift Suppression Events Are Required for Translation of Gag and Pol, 61 J. Virol. 480-490 (1987), and the human immunodeficiency virus type 1 (HIV-1) gag-pol frameshift, N. T. Parkin et al., Human Immunodeficiency Virus Type 1 Gag-Pol Frameshifting Is Dependent on Downstream mRNA Secondary Structure: Demonstration by Expression In Vivo, 66 J. Virol. 5147-5151 (1992).

Polyamines affect many biochemical processes within the cell. C. W. Tabor & H. Tabor, Polyamines, 53 Annu. Rev. Biochem. 749-790 (1984); O. Heby & L. Persson, Molecular Genetics of Polyamine Synthesis in Eukaryotic Cells, 15 Trends Biochem. Sci. 153-158 (1990); S. Cohen, A Guide to the Polyamines (Oxford University Press, New York 1998); K. Igarashi & K. Kashiwagi, Polyamines: Mysterious Modulators of Cellular Functions, 271 Biochem. Biophys. Res. Commun. 559-564 (2000). Elevated polyamine levels are associated with cellular proliferation and transformation; whereas polyamine depletion is known to inhibit cellular growth, and extreme depletion results in cell death. S. Iwata et al., Anti-tumor Activity of Antizyme which Targets the Ornithine Decarboxylase (ODC) Required for Cell Growth and Transformation, 18 Oncogene 165-172 (1999); S. Cohen, supra. Polyamines have been shown to act through general ionic interactions with nucleic acids, proteins, and phospholipids and are required for numerous processes including translation, transcription, and viral packaging. Some specific roles include binding to macromolecules such as tRNAs, A. Garcia et al., New Photoactivatable Structural and Affinity Probes of RNAs: Specific Features and Applications for Mapping of Spermine Binding Sites in Yeast tRNA(Asp) and Interaction of this tRNA with Yeast Aspartyl-tRNA Synthetase, 18 Nucleic Acids Res. 89-95 (1990); L. Frydman et al., Interactions between Natural Polyamines and tRNA: an N NMR Analysis, 89 Proc. Nat'l Acad. Sci. USA 9186-9190 (1992), and nucleosomes, H. R. Matthews, Polyamines, Chromatines Structure and Transcription, 15 Bioessays 561-566 (1993), and in gating the inward rectifier current of an ion channel, A. N. Lopatin et al, Potassium Channel Block by Cytoplasmic Polyamines as the Mechanism of Intrinsic Rectification, 372 Nature 366-369 (1994); K. Williams, Interactions of Polyamines with Ion Channels 325 Biochem. J. 289-297 (1997). Spermidine is also required for the formation of hypusine modification of eIF-5A, Y. B. Lee et al., Complex Formation between Deoxyhypusine Synthase and its Protein Substrate, the Eukaryotic Translation Initiation Factor 5A (eIF5A) Precursor, 340 Biochem. J. 340, 273-281 (1999), and is necessary for its nuclear export, G. Lipowsky et al., Exportin 4: a Mediator of a Novel Nuclear Export Pathway in Higher Eukaryotes, 19 EMBO J.4362-4371 (2000). Clearly, these ubiquitous molecules and the regulation of their intracellular content are essential for the normal maintenance of cellular growth and function.

Ornithine decarboxylase (ODC) is the first and rate limiting enzyme in the formation of putrescine, from which the polyamines, spermidine and spermine, are derived. S. Hayashi & Y. Murakami, Rapid and Regulated Degradation of Ornithine Decarboxylase, 306 Biochem J. 1-10 (1995); Y. Murakami et al., Cloning of Antizyme Inhibitor, a Highly Homologous Protein to Ornithine Decarboxylase, 271 J. Biol. Chem. 3340-3342 (1996). Regulation of polyamine levels by interference with ODC activity has important clinical implications. Overexpression of ODC in NIH 3T3 and rat fibroblast cell lines induces cellular transformation and rapidly progressing tumors in nude mice. M. Auvinen et al., Ornithine Decarboxylase Activity is Critical for Cell Transformation, 360 Nature 355-358 (1992); J. A. Moshier et al., Transformation of NIH/3T3 Cells by Ornithine Decarboxylase Overexpression, 53 Cancer Res. 2618-2622 (1993); A. Clifford et al., Role of Ornithine Decarboxylase in Epidermal Tumorigenesis, 55 Cancer Res. 1680-1686 (1995). ODC inhibitors such as difluoromethylornithine (DFMO) can reduce cellular proliferation and inhibit tumor formation. F. L. Meyskens, Jr. & E. W. Gerner, Development of Difluoromethylornithine (DFMO) as a Chemoprevention Agent, 5 Clin. Cancer Res. 945-951 (1999). In addition, an established use for DFMO is in the treatment of a disease of major consequence, West African sleeping sickness caused by Trypanosoma brucei. C. J. Bacchi & N. Yarlett, Effects of Antagonists of Polyamine Metabolism on African Trypanosomes, 54 Acta Trop. 225-236 (1993), and DFMO is currently being marketed in the United States as a medical treatment for female facial hair growth.

A naturally occuring regulator of ODC is mediated by a family of proteins called antizymes S. Hayashi et al., Ornithine Decarboxylase Antizyme: a Novel Type of Regulatory Protein, 21 Trends Biochem. Sci. 27-30 (1996). Antizyme 1 inhibits ODC by forming a complex, W. F. Fong et al., The Appearance of an Ornithine Decarboxylase Inhibitory Protein upon the Addition of Putrescine to Cell Cultures, 428 Biochim. Biophys. Acta 456-465 (1976); J. S. Heller et al., Induction of a Protein Inhibitor to Ornithine Decarboxylase by the End Products of its Reaction, 73 Proc. Nat'l Acad. Sci. USA 1858-1862 (1976), leading to degradation of ODC by the 26S proteosome without ubiquitination, Y. Murakami et al., Ornithine Decarboxylase is Degraded by the 26S Proteosome without Ubiquitination, 360 Nature 597-599 (1992); Y. Murakami et al., Destabilization of Ornithine Decarboxylase by Transfected Antizyme Gene Expression in Hepatoma Tissue Culture Cells, 267 J. Biol. Chem. 13138-13141 (1992); X. Li & P. Coffino, Degradation of Ornithine Decarboxylase: Exposure of the C-terminal Target by a Polyamine-inducible Inhibitory Protein, 13 Mol. Cell. Biol. 2377-2383 (1993); Y. Murakami et al., Degradation of Ornithine Decarboxylase by the 26S Proteosome, 267 Biochem. Biophys. Res. Commun. 1-6 (2000). Like antizyme 1, both antizyme 2 and antizyme 3 inhibit ODC, and antizyme 2 has been shown to lead to increased degradation of ODC in cells. I. P. Ivanov et al., A Second Mammalian Antizyme: Conservation of Programmed Ribosomal Frameshifting, 52 Genomics 119-129 (1998); C. Zhu et al., Antizyme 2 Is a Negative Regulator of Ornithine Decarboxylase and Polyamine Transport, 274 J. Biol. Chem. 26425-26430 (1999). Antizyme 3 appears to play a tissue specific role in polyamine regulation as its mRNA is only transcribed in germ cells during the later stages of spermatogenesis, I. P. Ivanov et al., Discovery of a Spermatogenesis Stage-specific Ornithine Decarboxylase Antizyme: Antizyme 3, 97 Proc. Nat'l Acad. Sci. USA 97, 4808-4813 (2000); Y. Tosaka et al., Identification and Characterization of Testis Specific Ornithine Decarboxylase Antizyme (OAZ-t) Gene: Expression in Haploid Germ Cells and Polyamine-induced Frameshifting, 5 Genes Cells 265-276 (2000), while antizyme 1 and 2 mRNAs have a nearly ubiquitous tissue distribution, S. Matsufuji et al., Analyses of Ornithine Decarboxylase Antizyme mRNA with a cDNA Cloned from Rat Liver, 108 J. Biochem. (Tokyo) 365-371 (1990). In addition, antizyme 1 mRNA has a mitochondrial localization signal near the amino terminus that is lacking in both antizyme 2 and antizyme 3 mRNA, implying a role in polyamine regulation within the mitochondria. J. L. Mitchell & G. G. Judd, Antizyme Modifications Affecting Polyamine Homocostasis, 26 Biochem. Soc. Trans. 591-595 (1998). Finally, antizymes 1 and 2 have also been shown to play a role in polyamine transport by inhibiting polyamine uptake into the cell and stimulating polyamine export J. L. Mitchell et al, Feedback Repression of Polyamine Transport Is Mediated by Antizyme in Mammalian Tissue-culture Cells, 299 Biochem J. 19-22 (1994); T. Suzuki. et al, Antizyme Protects against Abnormal Accumulation and Toxicity of Polyamines in Ornithine Decarboxylase-overproducing Cells, 91 Proc. Nat'l Acad. Sci. USA 8930-8934 (1994); K. Sakata et al., Identification of Regulatory Region of Antizyme Necessary for the Negative Regulation of Polyamine Transport, 238 Biochem Biophys. Res. Commun. 415-419 (1997); K. Stakata et al., Properties of a Polyamine Transporter Regulated by Antizyme, 347 Biochem. J. 297-303 (2000). Thus, antizyme proteins affect intracellular polyamine levels by regulating both the ODC biosynthetic pathway and polyamine transport into and out of the cell.

The level of antizyme mRNA is high even when no protein is detectable, which is in agreement with the antizyme gene carrying a strong constitutively expressed promoter. S. Matsufuji et al., Monoclonal Antibody Studies on the Properties and Regulation of Murine Ornithine Decarboxylase Antizymes, 107 J. Biochem. (Tokyo) 87-91 (1990); Y. Miyazaki et al., Cloning and Characterization of a Rat Gene Encoding Ornithine Decarboxylase Antizyme, 113 Gene 191-197 (1992). Antizyme levels are regulated post-transcriptionally by an unusual translational frameshift mechanism. Antizyme genes contain two overlapping open reading frames (ORFs) with the second downstream ORF in the +1 reading frame relative to the upstream ORF such that a +1 translation frame shift event is required for the production of full length active antizyme protein. Frameshifting of antizyme 1 occurs at a specific site and is stimulated by an adjacent stop codon in the 0 frame, as well as RNA sequences 5′ and an RNA pseudoknot 3′ of the shift site. S. Matsufuji et al., Autoregulatory Frameshifting in Decoding Mammalian Ornithine Decarboxylase Antizyme, 80 Cell 51-60 (1995). Using an in vitro rabbit reticulate lysate translation system, it has been shown that full length antizyme 1 and antizyme 2 are produced by a +1 translation shift that is stimulated by elevated polyamine levels. Antizyme 3 is likely to have a similar regulatory mechanism.

Antizyne 1 binds specifically to at least one other protein in addition to ODC (and probably the polyamine transporter). When antizyme 1 binds to a protein termed antizyme inhibitor, its ability to bind and inhibit ODC is prevented. K. Fujita et al., A Macromolecular Inhibitor of the Antizyme to Ornithine Decarboxylase, 204 Biochem. J. 647-652 (1982); K. Koguchi et al., Cloning and Sequencing of a Human cDNA Encoding Ornithine Decarboxylase Antizyne Inhibitor, 1353 Biochim Biophys. Acta 209-216 (1997); C. Koike et al, Sensitivity to Polyamine-induced Growth Arrest Correlates with Antizyme Induction in Prostate Carcinoma Cells, 59 Cancer Res. 6109-6112 (1999); J. Nilsson et al, Antizyme Inhibitor Is Rapidly Induced in Growth-stimulated Mouse Fibroblasts and Releases Ornithine Decarboxylase from Antizyme Suppression, 346 Biochem. J. 699-704 (2000); R. C. Smith et al, identification of an Endogenous Inhibitor of Prostatic Carcinoma Cell Growth, 1 Nat. Med. 1040-1045 (1995). Whether antizyme inhibitor has additional functions is unknown at this time.

Based on the evidence described above, it has been proposed that antizyme frameshifting is an intracellular sensor for polyamine level that controls antizyme expression. Once produced, antizyme activity is further modulated by antizyme inhibitor to tightly regulate the ODC biosynthetic pathway and polyamine transport into and out of the cell. J. Satriano et al., Agmatine Suppresses Proliferation by Frameshift Induction of Antizyme and Attenuation of Cellular Polyamine Levels, 273 J. Biol. Chem. 15313-15316. (1998); S. Vujcic et al., Effects of Conditional Overexpression of Spermidine/Spermine N1-acetyltransferase on Polyamine Pool Dynamics, Cell Growth, and Sensitivity to Polyamine Analogs, 275 J. Biol. Chem. 38319-38328 (2000); R. A. Casero, Jr. & A. E. Pegg, Spermidine/spermine N1-acetyltransferase—the Turning Point in Polyamine Metabolism, 7 FASEB J. 653-661 (1993).

It is noteworthy that none of the prior art known to the present applicants provides an assay or compositions and methods that can be used to perform a quantitative analysis of polyamines, polyamine analogues, and other frameshift analogues in cells. The present invention provides a tissue culture assay for measuring drug induced recoding in regulating cellular polyamines by utilizing the methods and components described herein.

In view of the foregoing, it will be appreciated that providing a quantitative method for the analysis of polyamines, polyamine analogues, and other frameshift agonists in cells would be a significant advancement in the art. It will also be appreciated that providing an assay for measuring specific ribosomal frameshifting would also be a significant advancement in the art. It will further be appreciated that providing DNAs that can be used to quantitatively determine the occurrence of translational frameshifting in cell lines in which such DNAs have been provided would be another significant advancement in the art.

### BRIEF SUMMARY OF THE INVENTION

An illustrative embodiment of the present invention comprises an assay for quantitatively measuring drug-induced recoding in regulating cellular polyamine levels in cells. A DNA construct is provided containing the renilla luciferase gene separated by a short cloning site from the firefly luciferase gene, both under the control of a single upstream promoter functional in mammalian cells, such as an SV40, cytomegalovirus (CMV), or eukaryotic polymerase II promoter. The cloning site contains a portion of antizyme gene known to contain the mRNA signals for polyamine stimulated frameshifting with the downstream firefly gene in the +1 position relative to the upstream renilla gene. A control consist is also produced with the genes in the same reading frame. Frameshifting efficiencies can be determined by comparing the ratio of firefly to renilla enzymatic activities in parallel cell cultures.

Another illustrative embodiment of the present invention comprises a plasmid for use in assaying cellular polyamine levels comprising:

(a) an upstream DNA segment comprising a first open reading frame encoding a first reporter,

(b) a downstream DNA segment comprising a second open reading frame encoding a second reporter;

(c) a promoter positioned and operative for promoting transcription of the upstream and downstream DNA segments; and

(d) a polyamine-regulated frameshifting sequence positioned between the upstream DNA segment and the downstream DNA segment such that the second open reading frame is in a +1 reading frame with respect to the first open reading frame;

wherein, after transfection of the plasmid into cultured mammalian cells, an effective amount of polyamine stimulates +1 translational frameshifting, thereby resulting in increased expression of the second reporter as compared to expression of the first reporter. In one embodiment of this invention, the first reporter comprises renilla luciferase and the second reporter comprises firefly luciferase. In another illustrative embodiment of the invention, however, the first reporter comprises firefly luciferase and the second reporter comprises renilla luciferase. Illustrative polyamine-regulated frameshifting sequences that can be used comprise an antizyme 1 or an antizyme 2 polyamine-regulated frameshifting sequence, such as any of SEQ ID NO:1 through SEQ ID NO:6, or an antizyme 3 polyamine-regulated frameshifting sequence.

Another illustrative embodiment of the invention comprises a plasmid for use in assaying cellular polyamine levels comprising:

(a) an upstream DNA segment comprising a first open reading frame encoding a first reporter;

(b) a downstream DNA segment comprising a second open reading fine encoding a second reporter;

(c) a promoter positioned and operative for promoting transcription of the upstream and downstream DNA segments; and

(d) a polyamine-regulated frameshifting sequence positioned between the upstream DNA segment and the downstream DNA segment such that the second open reading frame is in a different reading frame with respect to the first open reading frame;

wherein, after transfection of the plasmid into cultured mammalian cells, an effective amount of polyamine stimulates translational frameshifting such that the first open reading frame and the second open reading frame are translated in the same frame, thereby resulting in increased expression of the second reporter as compared to expression of the first reporter.

Still another illustrative embodiment of the invention comprises a method of estimating recoding in genes involved in regulating cellular polyamine levels comprising:

(a) transfecting a first set of cultured mammalian cells with a first dual reporter assay construct comprising

(i) an upstream DNA segment comprising a first open reading frame encoding a first reporter,

(ii) a downstream DNA segment comprising a second open reading frame encoding a second reporter,

(iii) a promoter positioned and operative for promoting transcription of the upstream and downstream DNA segments, and

(iv) a polyamine-regulated frameshifting sequence positioned between the upstream DNA segment and the downstream DNA segment such that the second open reading frame is in a +1 reading frame with respect to the first open reading frame;

(b) transfecting a second set of cultured mammalian cells with a second dual reporter assay construct comprising the first dual reporter assay construct except that the second open reading frame is in the same reading frame with respect to the first open reading frame;

(c) growing the transfected first set of cultured mammalian cells and the transfected second set of cultured mammalian cells and determining a ratio of levels of expression of the second reporter compared to the first reporter in each of the first set and the second set of cultured mammalian cells; and

(d) comparing each ratio, wherein a proportion of the ratio for the first set of cultured mammalian cells to the ratio for the second set of cultured mammalian cells is an estimate of recoding in the genes involved in regulating cellular polyamine levels. In another illustrative embodiment of this inventions this method further comprises treating the first and second sets of cultured mammalian cells such that endogenous levels of polyamines arm reduced. The endogenous polyamine levels can be reduced by any of several approaches, such as treating the cells with an inhibitor of polyamine biosynthesis or a stimulator of polyamine excretion or catabolism. Illustrative inhibitors of polyamine biosynthesis include inhibitors of ornithine decarboxylase, such as difluoromethylornithine (DFMO), and inhibitors of S-adenosyl methionine decarboxylase. An illustrative simulator of polyamine excretion or catabolism comprises spermidine/spermine N′-acetyltransferase (SSAT).

Still another illustrative embodiment of the present invention comprises a method for screening for small molecules that affect frameshifting in cells, comprising:

(a) transfecting a first set of cultured mammalian cells with a first dual reporter assay construct comprising

(i) an upstream DNA segment comprising a first open reading frame encoding a first reporter,

(ii) a downstream DNA segment comprising a second open reading frame encoding a second reporter,

(iii) a promoter positioned and operative for promoting transcription of the upstream and downstream DNA segments, and

(iv) a polyamine-regulated frameshifting sequence positioned between the upstream DNA segment and the downstream DNA segment such that the second open reading frame is in a different reading frame with respect to the first open reading frame;

(b) transfecting a second set of cultured mammalian cells with a second dual reporter assay construct comprising the first dual reporter assay construct except that the second open reading frame is in the same reading frame with respect to the first open reading frame;

(c) growing the first set of cultured mammalian cells and the second set of cultured mammalian cells in the presence of a candidate small molecule and determining a ratio of levels of expression of the second reporter compared to the first reporter for each of the first set and the second set of cultured mammalian cells; and

(d) comparing each said ratio, wherein an increase or decrease in the ratio indicates that the small molecule affects frameshifting.

Still another illustrative embodiment of the present invention comprises a method for screening for small molecules that affect polyamine regulation in cells, comprising:

(a) transfecting a first set of cultured mammalian cells with a first dual reporter assay construct comprising

(i) an upstream DNA segment comprising a first open reading frame encoding a first reporter,

(ii) a downstream DNA segment comprising a second open reading fame encoding a second reporter,

(iii) a promoter positioned and operative for promoting transcription of the upstream and downstream DNA segment; and

(iv) a polyamine-regulated frameshifting sequence positioned between the upstream DNA segment and the downstream DNA segment such that the second open reading frame is in a +1 reading frame with respect to the first open reading frame;

(b) transfecting a second set of cultured mammalian cells with a second dual reporter assay construct comprising the first dual reporter assay construct except that the second open reading frame is in the same reading frame with respect to the first open reading frame;

(c) growing the first set of cultured mammalian cells and the second set of cultured mammalian cells in the presence of a candidate small molecule and determining a ratio of levels of expression of the second reporter compared to the first reporter for each of the first set and the second set of cultured mammalian cells; and

(d) comparing each said ratio, wherein an increase or decrease in the ratio indicates that the small molecule affects polyamine regulation.

Yet another illustrative embodiment of the present invention comprises a method for screening for small molecules that affect polyamine regulation in cells, comprising:

(a) transfecting a first set of cultured mammalian cells with a first dual reporter assay construct comprising

(i) an upstream DNA segment comprising a first open reading frame encoding a first reporter,

(ii) a downstream DNA segment comprising a second open reading frame encoding a second reporter,

(iii) a promoter positioned and operative for promoting transcription of the upstream and downstream DNA segments, and

(iv) a polyamine-regulated frameshifting sequence positioned between the upstream DNA segment and the downstream DNA segment such that the second open reading frame is in a +1 reading frame with respect to the first open reading frame;

(b) transfecting a second set or cultured mammalian cells with a second dual reporter assay construct comprising the first dual reporter assay construct except that the second open reading frame is in the same reading frame with respect to the first open reading frame;

(c) treating the first set of cultured mammalian cells and the second set of cultured mammalian cells such that endogenous levels of polyamine are reduced;

(d) growing the fist set of cultured mammalian cells and the second set of cultured mammalian cells in the presence of a candidate small molecule and determining a ratio of levels of expression of the second reporter compared to the first reporter for each of the first set and the second set of cultured mammalian cells; and

(e) comparing each ratio, wherein an increase or decrease in the ratio indicates that the small molecule affects polyamine regulation.

Additional embodiments and advantages of the invention will be set forth in the description that follows, and in part will be apparent from the description, or may be learned by the practice of the invention without undue experimentation. The embodiments and advantages of the invention may be realized and obtained by means of the instruments and combinations particularly pointed out in the appended claims.

### BRIEF DESCRIPTION OF THE SEVERAL VIEWS OF THE DRAWINGS

FIG. 1 is a schematic diagram showing the synthesis, transport, and regulation of polyamines by antizyme; the curved arrow represents control by polyamines of the frequency of a +1 translational frameshift in antizyme genes, lines with bars at the ends represent antizyme inhibition of ODC and polyamine transport, ODC=ornithine decarboxylase, Orf=open reading frame, ADC=arginine decarboxylase.

FIG. 2 is a schematic representation of the SV40 promoter, renilla luciferase and firefly luciferase reporter genes, and multiple cloning site of the frameshift reporter plasmid, p2Luc.

FIGS. 3A-C are bar graphs representing antizyme 1 frameshifting in tissue culture cells in response to the presence (gray bars) or absence (black bars) of DFMO and putrescine (FIG. 3A), spermidine (FIG. 3B), or spermine (FIG. 3C).

FIG. 4 is a bar graph representing antizyme 2 frameshifting in a mammalian cell line in response to the presence (gray bars) or absence (black bars) of DFMO and spermidine.

FIG. 5 is a bar graph showing the effects of deletion of the upstream and downstream antizyme 1 and 2 frameshift stimulatory sequences on frameshifting in mammalian cells in the absence of polyamine and DFMO (None), in the presence of 1 mM spermidine (Spd), in the presence of 2.5 mM DFMO (DFMO), and in the presence of 1 mM spermidine and 2.5 mM DFMO (DFMO+Spd); the constructs used were p2Lucaz1pkdel (black bars), p2Lucaz2pdkel (light gray bars) p2Lucaz1usdel (dark gray bars), p2Lucaz2usdel (white bars).

FIG. 6 is a bar graph showing the effect of extracellular polyamine addition on intracellular polyamine levels measured in cells grown in standard growth media (None), and growth media supplemented with 1 mM spermidine (Spd), 2.5 mM DFMO (DFMO), or with both DFMO and spermidine (DFMO+Spd); the concentration of measured putrescine (black bars), spermidine (light gray bars), and spermine (medium gray bars) is shown as nmol/10⁶ cells.

FIGS. 7A-B are bar graphs showing the effects of extracellular agmatine (0-16 mM; FIG. 7A) and cadaverine (0-32 mM; FIG. 7B) addition on frameshifting in cells containing an antizyme 1 (medium gray bars), antizyme 2 (hatched bars), or antizyme 3 (black bars) polyamine-regulated frameshifting sequence; controls were exposed to 2.5 mM DFMO.

### DETAILED DESCRIPTION

Before the present assay for measuring drug-induced recoding and compositions associated therewith are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a composition containing “a polyamine” includes a mixture of two or more of such polyamines, reference to “an ODC inhibitor” includes reference to one or more of such ODC inhibitors, and reference to “an antizyme” includes references to two or more of such antizymes.

In describing and claiming the present invention, the following terminology will be used in accordance with the definitions set out below.

As used herein, “comprising,” “including,” “containing,” “characterized by,” and grammatical equivalents thereof are inclusive or open-ended term that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “ODC” means ornithine decarboxylase, “ADC” means arginine decarboxylase, and “ORF” means open reading frame.

As used herein, “transfection,” “transfecting,” and similar terms are intended to include both stable transfection and transient transfection.

As used herein, “polyamine-regulated frameshifting sequence” means a frameshifting or shift site and, optionally, associated 5′ and/or 3′ regulatory signals that affect the efficiency of frameshifting. For example, the antizyme 1 polyamine-regulated frameshifting sequence of SEQ ID NO:1 includes a frameshifting site (TCCT) at nucleotides 55-58, an upstream or 5′ regulatory signal, and a downstream or 3′ pseudoknot regulatory signal. By way of further example, the polyamine-regulated frameshifting sequence of SEQ ID NO:3 contains a shift site (TCCT) at nucleotides 10-13 and a 3′ pseudoknot regulatory signal, but contains no 5′ regulatory signal. By way of still further example, the polyamine-regulated frameshifting sequence of SEQ ID NO:5 contains a shift site (TCCT) at nucleotides 55-58 and a 5′ regulatory signal, but contains no 3′ regulatory signal.

As used herein, “reporter” means a gene product that can be assayed for determining the amount of gene product produced in a cell containing a DNA coding for the reporter. For example, reporters used in an illustrative embodiment of the present invention include renilla and firefly luciferases. Other reporters that can be used according to the present invention include β-galactosidase (β-gal), glutathione S-transferase (GST), chloramphenicol acetyl transferase (CAT), green fluorescent protein (GFP) and derivatives thereof such as YFP and BFP, horseradish peroxidase (HRP), and alkaline phosphatase, and the like. In addition, other possible reporters according to the present invention include proteins that are recognized by secondary molecules. An example of such a reporter is streptavidin, which can be quantified by measuring the amount of binding to a biotin-linked enzyme, wherein the enzyme can be readily assayed, such as alkaline phosphatase. Another example of such a reporter is protein A, which can be quantified by measuring the amount of binding to an antibody-linked enzyme, wherein the enzyme can be readily assayed.

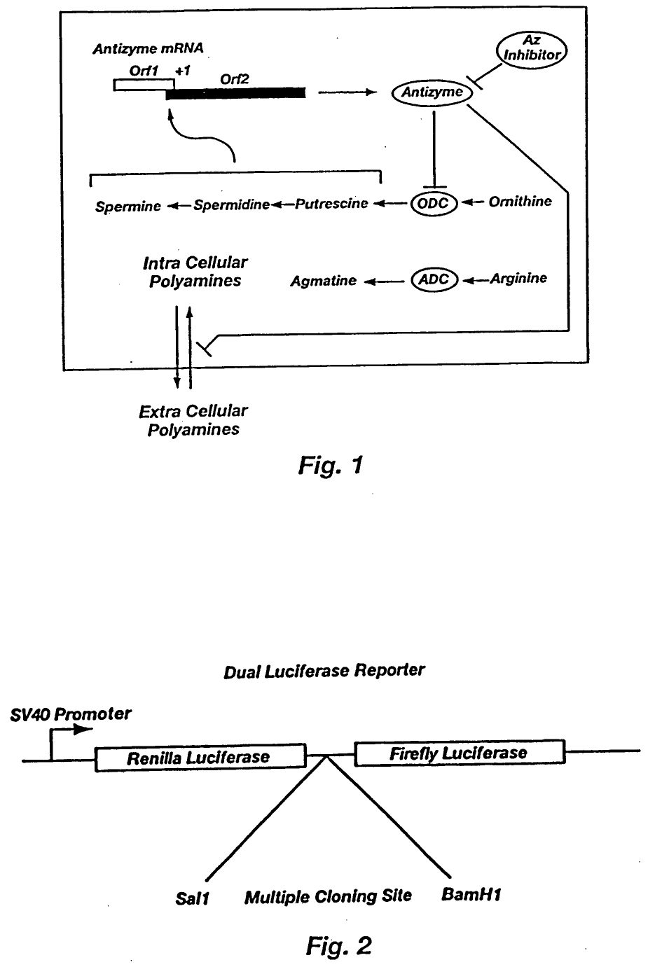

As reviewed above, antizyme is a critical regulation of cellular polyamine levels due to its effect on polyamine transport and it ability to target ODC for degradation. Antizyme expression depends upon an unusual +1 translational frameshift mechanism that is regulated by polyamine levels to form an autoregulatory loop. FIG. 1 is a schematic diagram depicting the regulation of polyamine levels by the antizyme regulatory loop. Intracellular polyamines control the frequency of a +1 translational frameshift in antizyme genes (indicated by the curved arrow). High levels of the polyamines putrescine, spermidine, and/or spermine cause an increase in antizyme frameshifting resulting in an increase in full length active antizyme protein. Antizyme protein inhibits ODC and polyamine transport (indicated by the lines with bars at the end). The reduction of intracellular polyamine levels resulting from the antizyme activity consequently lowers antizyme levels, completing an autoregulatory loop.

As disclosed herein, it has been discovered that a dual reporter assay can quantitatively measure the effect and levels of polyamines on tissue culture cells. The assay comprises placing a polyamine-regulated frameshifting sequence between two DNAs that code for reporter proteins, wherein the open reading frame of the downstream reporter DNA is in the +1 reading frame as compared to the open reading frame of the upstream reporter DNA. In an illustrative embodiment of the invention, two dual reporter assay constructs are created by placing a portion of an antizyme gene known to contain the mRNA signals for polyamine-stimulated frameshifting between an upstream DNA encoding a first reporter and a downstream DNA encoding a second reporter. The first such construct places the ORF of the downstream DNA in the +1 reading frame relative to the ORF of the upstream DNA. With this first construct, the second reporter is expressed only when frameshifting (recoding) takes place. The second (control) construct places the ORF of the downstream DNA in the same or “0” frame relative to the ORF of the upstream DNA. With this second construct, no frameshifting (recoding) is required for expression of the second reporter to occur. Parallel cell lines are transfected with the two constructs such that transcription and translation proceed in vivo. By comparing the levels of the protein or enzyme activity resulting from expression of the first and second reporters in the parallel cultures, the levels of polyamine activity can be quantitatively assessed. Activity of the first reporter provides an internal control for normalizing differences in transfection efficiencies, translation initiation, and mRNA stability.

Referring now to FIG. 2, the construction of illustrative embodiments of the dual reporter assay constructs is depicted. These illustrative embodiments use the renilla and firefly luciferases as first and second reporters, respectively. The sequences referred to herein as SEQ ID NO:1 and SEQ ID NO:2, which comprise antizyme 1 and 2 polyamine-regulated frameshifting sequences, respectively, were cloned into the SalI and BamHI sites of the p2Luc vector (U.S. Pat. No. 6,143,502) to produce plasmids p2Lucaz1 and p2Lucaz2. The shift site (TCCT), corresponding to UCCU in the corresponding mRNA, is present at nucleotides 55-58 of each of these sequences. Constructs were also prepared with deletions of the upstream stimulatory sequences of the antizyme 1 polyamine-regulated frameshifting sequence (SEQ ID NO:3) and the antizyme 2 polyamine-regulated frameshifting sequence (SEQ ID NO:4), resulting in plasmids p2Lucaz1usdel and p2Lucaz2usdel. Other constructs were prepared with deletions of the downstream pseudoknot stimulatory sequences of the antizyme 1 polyamine-regulated frameshifting sequence (SEQ ID NO:5) and the antizyme 2 polyamine-regulated frameshifting sequence (SEQ ID NO:6), resulting in plasmids p2Lucaz1pkdel and p2Lucaz2pkdel. Zero frame (“0 frame”) controls for each construct were identical except that the first T of the stop codon following the shift site was deleted. It will be appreciated that the DNA constructs disclosed herein are merely examples of dual reporter constructs comprising two reporter-encoding DNA segments separated by a cloning site containing a polyamine-regulated frameshifting sequence, and that all such constructs are included within the scope of the present invention.

An illustrative method of constructing the dual reporter constructs of FIG. 2 is by synthesis of the sequences necessary and sufficient for translational frameshifting (the sequences of the top strand of illustrative embodiments are shown in SEQ ID NO:1 through SEQ ID NO:6) through the addition of complementary oligonucleotides, such that when annealed the strands will have SalI and BamHI compatible ends. These oligonucleotides can be synthesized according to methods well known in the art, such as with an automated synthesizer (e.g., Applied Biosystems model 380C). It will be appreciated that any available method for synthesizing the dual reporter constructs may be used and all such methods known now, or in the future, to those skilled in the art are within the scope of the present invention. The frameshift sequences are then ligated into SalI and BamHI digested p2Luc vector (G. Grentzmann et al., A Dual-luciferase Reporter System for Studying Recoding Signals, 4 RNA 479-486 (1998); U.S. Pat. No. 6,143,502). These may be amplified by transformation into E. coli strain SU1675, although any suitable amplification mechanism may be used. It is advantageous to verify that the construct sequences by autothermocycler sequencing of a sample prior to use.

Cultured mammalian cells are transfected with the dual construct according to method well known in the art. Parallel cell lines are transfected with the +1 reading frame and 0 reading frame construct according to methods well known in the art. The cells are then incubated under varying conditions to test the effect of such conditions on the polyamine regulation. Cells are then lysed and the luciferase activity measured by light emission after injection of luminescence substrate, as is well known in the art. The ratio of firefly to renilla luciferase activity in cultures transfected with the +1 frame construct is compared to the ratio of those transfected with the 0 frame construct as described in Grentzmann, supra. This provides a quantitative assay for measuring the polyamine-induced recoding.

The effects of inhibiting polyamine biosynthesis and/or stimulating polyamine catabolism may thus be determined using this system, as illustrated in the following examples. These examples are merely illustrative and are not intended to limit the scope of the invention.

### EXAMPLE 1

Human embryonic kidney (HEK293) cells were grown as monolayer cultures in Dulbecco's Modified Eagle Media (DMEM) with 1,000 mg/L D-glucose, L-glutamine, and pyridoxine hydrochloride, and 110 mg/L sodium pyruvate supplemented with 10% fetal bovine serum (FBS) and 50 units/ml penicillin/50 μg/ml streptomycin. Similarly, mouse germ line (GC-2) cells were obtained from the American Tissue Type culture Collection (Manassas, Va.). These GC-2 cells had been maintained as a monolayer in DMEM with 4,500 mg/L D-glucose and L-glutamine, 110 mg/L sodium pyruvate and pyridoxine hydrochloride supplemented with 10% FBS, 50 units/ml penicillin/50 μg/ml streptomycin, and 1% nonessential amino acids. All cells were incubated at 37° C. in an atmosphere of 5% CO₂ All media and antibiotics were obtained from Invitrogen Life Technologies (Carlsbad, Calif.), and all sera were obtained from HyClone Inc. (Logan Utah).

The cells were transfected with the dual luciferase constructs p2Lucaz1 and p2Lucaz2 using LIPOFECTIN reagent (Invitrogen Life Technologies). LIPOFECTIN reagent is a 1:1 (w/w) liposome formulation of the cationic lipid N-[1-(2,3-dioleyloxy)propyl]-n,n,n,-trimethylammonium chloride (DOTMA), and dioleoyl phosphotidylethanolamine (DOPE) in membrane filtered water. Cells (0.3×10⁵) were plated in 48-well, treated tissue culture plates and grown for 48 hours as described above. DFMO (2.5 mM) was added to half the plates of each cell line to inhibit polyamine synthesis. All cells were transfected with 0.6 μl LIPOFECTIN reagent and 0.2 μg plasmid DNA (as described above) for 15 hours in serum free media in the presence or absence of DFMO. The fresh media with serum, 1 mM aminoguanidine, and varying levels of polyamines or DFMO were added and incubation continued for 12 hours prior to analysis.

Cells were lysed using passive lysis buffer and luciferase activity was determined using the Dual Luciferase reporter assay (Promega, Madison, Wis.) as described in Grentzmann, supra. For all reactions, light emission was measure between 2 and 12 seconds after 100 μl of luminescence substrate was injected. Frameshift efficiency was calculated by comparing the ratio of firefly to luciferase activity in cultures transfected with p2Luc antizyme constructs and compared to ratios obtained from cultures transfected with p2Luc in-frame control constructs as described in Grentzmann, supra.

The endogenous antizyme levels were also measured for comparison. HEK293 cells were plated in 10 cm dishes with 10 ml of the growth media as described above at a concentration of 3×10⁵ cells/ml and incubated for 24 hours. The medium was replaced with fresh medium supplemented with 1 mM aminoguanidine and various concentrations of either putrescine, spermidine, or spermine. The dishes were incubated further for 24 hours, placed on ice, washed with phosphate buffered saline twice, drained completely, and cells were disrupted by 3 freeze/thaw cycles. Then 0.5 ml of cell extract buffer (25 mM Tris-HCl, pH 7.2, 1 mM dithiothreitol, 1 mM EDTA, and 0.01% Tween 80) was added, and the cell suspension was centrifuged at 15,000 rpm for 30 minutes at 4° C. Antizyme activities were measured as ODC-inhibitory activities that could be reversed by excess antizyme inhibitor. Cell extracts (40 μl) were mixed with 2.0 units of mouse kidney ODC in duplicate. To one set, 0.1 μg of GST-antizyme inhibitor protein was added. ODC activity of each mixture was assayed by measuring the release of ¹⁴CO₂ from L-[1-¹⁴C] ornithine (as described in I. P. Ivanov et al., Discovery of a Spermatogenesis Stage-specific Ornithine Decarboxylase Antizyme: Antizyme 3, 97 Proc. Nat'l Acad. Sci. USA 97, 4808-4813 (2000); I. P. Ivanov et al., Conservation of Polyamine Regulation by Translational Frameshifting from Yeast to Mammals, 19 EMBO J. 1907-1917 (2000); S. Matsufuji et al., Reading Two Bases Twice: Mammalian Antizyme Frameshifting in Yeast, 15 EMBO J. 1360-1370 (19%)), and expressed as specific activities. Protein concentrations of the extracts were determined with the BCA protein assay kit (Pierce) using bovine serum albumin as a standard. One unit of ODC and antizyme was defined as the activity releasing 1 nM CO₂ per hour and the activity inhibiting 1 unit of ODC, respectively.

Polyamine levels were also measured for comparison. Intracellular polyamine levels were determined by HPLC analysis of whole cell lysates. Cells (3×10⁵) were grown in 6-well plates for 48 hours at 37° C. in an atmosphere of 5% CO₂, either in the presence or absence of DFMO. Cells were then mock transfected as described above. Following transfection, the medium was exchanged for DMEM containing 10% FBS with the addition of 2.5 mM DFMO and 1 mM spermidine as indicated. Cells were allowed to grow for 12 hours, and 5×10⁶ cells were harvested and washed three times with phosphate buffered saline. Cells were lysed by perchloric acid and dansylated prior to HPLC polyamine analysis (as described in P. M. Kabra et al., Solid-phase Extraction and Determination of Dansyl Derivatives of Unconjugated and Acetylated Polyamines by Reversed-phase Liquid Chromatography: Improved Separation Systems for Polyamines in Cerebrospinal Fluid, Urine and Tissue, 380 J. Chromatogr. 19-32 (1986)).

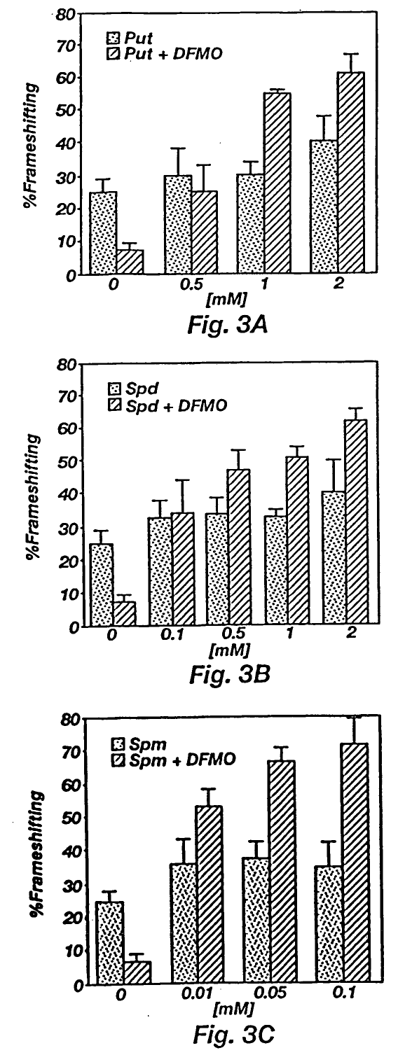

Results of this experiment show the present invention to provide an accurate measurement of frameshifting in response to polyamine levels in HEK293 cells grown under various conditions designed to deplete or increase those levels. Under standard growth conditions (DMEM supplemented with 10% FBS), approximately 25% of translating ribosomes shifted to the +1 frame. Upon addition of exogenous putrescine, spermine, or spermidine, a small increase in frameshifting was observed using the dual luciferase reporter system, as shown in FIGS. 3A, 3B and 3C. Maximal frameshifting stimulation of approximately 40%, a 1.3 to 1.5 fold increase, was observed at concentrations of 0.01-0.1 mM spermine, 0.1-2 mM spermidine, and 0.5-2 mM putrescine. Similar results were obtained by measuring endogenous antizyme levels as ODC inhibitory activity in cell extracts (Table 1). Endogenous antizyme levels increased 1.1-fold to 1.6-fold upon the addition of exogenous spermine, spermidine, and putrescine. It follows that under standard growth conditions, antizyme frameshifting is relatively insensitive to exogenous polyamine addition in HEK293 cells. With this embodiment of the present invention this result was demonstrated as efficiently as with the traditional excess antizyme inhibiting test, Y. Murakami et al., Cloning of Antizyme Inhibitor, a Highly Homologous Protein to Ornithine Decarboxylase, 271 J. Biol. Chem. 3340-3342 (1996).

TABLE 1

| Treatment | Antizyme (units/mg) | Fold Increase |
|---|---|---|
| Control | 2.1 ± 0.9 | 1.0 |
| Putrescine 2 mM | 3.0 ± 0.7 | 1.5 |
| Spermidine 1 mM | 23 ± 0.2 | 1.1 |
| Spermidine 2 mM | 3.4 | 1.6 |
| Spermine 0.4 mM | 2.6 ± 0.5 | 1.2 |
| Spermine 0.8 mM | 3.0 ± 0.6 | 1.4 |

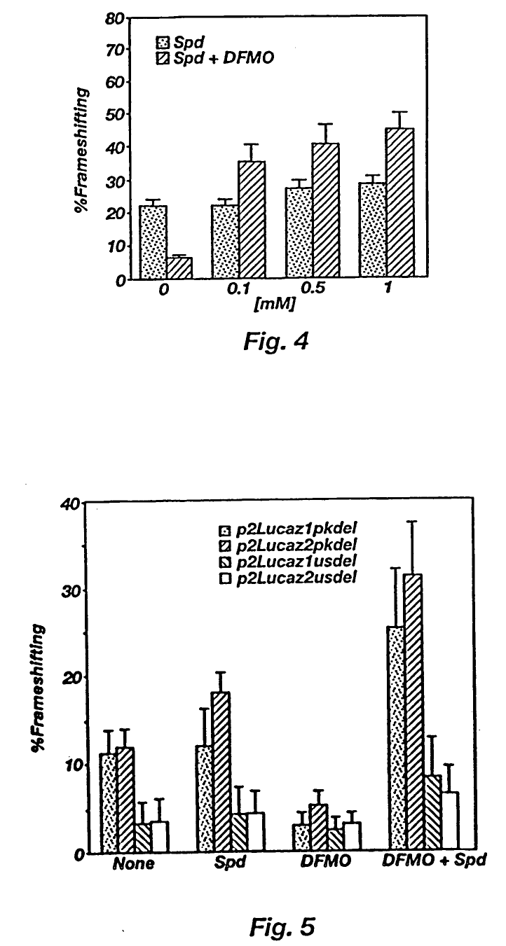

The present invention also accurately determined the extent to which HEK293 cells pre-treated with DFMO were more sensitive to exogenous polyamine addition with regard to the frequency of both antizyme 1 and antizyme 2 frameshifting. Antizyme 1 frameshifting levels were measured at approximately 6% after treatment with DFMO for 48 hours and increased nearly 10-fold to a maximum of between 60% and 70% upon addition of 0.1 mM spermine, 2 mM spermidine, and 2 mM putrescine (FIGS. 3A-C). Antizyme 2 frameshifting, although slightly lower, showed a very similar response to polyamine depletion and addition, as shown for spermidine in FIG. 4. The low levels of endogenous antizyme that is not inhibited by DFMO under these conditions made it impossible to measure endogenous antizyme activity levels using the ODC activity as described above. Efforts to measure the endogenous antizyme levels via a western blot were also unsuccessful due to the very low levels of endogenous antizyme. Previous reports indicate that antizyme is present at or below 1 ppm. This demonstrates the effectiveness of the present invention in measuring antizyme activity in conditions where traditional methods cannot.

### EXAMPLE 2

To determine the effectiveness of the role of sequences flanking the frameshift site in inducing frameshifting levels, dual luciferase constructs were made in which antizyme sequences preceding and following the antizyme 1 and 2 frameshift sites were deleted. As described above, p2Lucaz1usdel and p2Lucaz2usdel correspond to the deletion of upstream antizyme sequences. Likewise, p2Lucaz1pkdel and p2Lucaz2pkdel correspond to deletions of sequences downstream from the frameshift site that form a pseudoknot in the mRNA. As described above, these constructs were then transiently transfected into HEK293 cells grown in standard growth nedia or media with 2.5 mM DFMO, and frameshift levels were measured following the addition of exogenously added spermidine (1 mM). In all cases, exogenous spermidine addition resulted in increased frameshift levels. The upstream deletion construct were stimulated 5-6 fold by the addition of 1 mM spermidine to DFMO treated cells whereas the downstream (pseudoknot) deletions were stimulated approximately 2-fold These results demonstrate the functionality of the present invention, using alterative embodiments.

### EXAMPLE 3

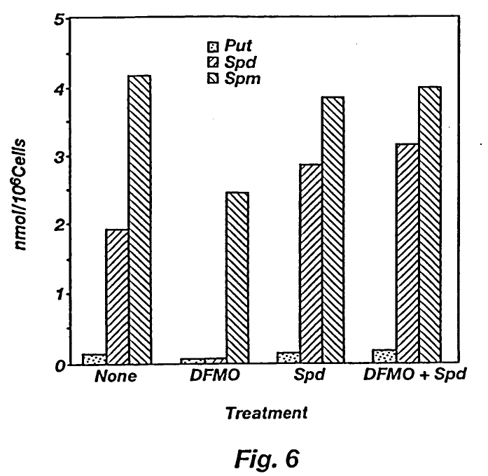

To measure the effect of DFMO pre-treatment on the intracellular polyamine levels of tissue culture cells, HEK293 cells were grown, as described above, either in the presence or absence of DFMO (2.5 mM) for 48 hours followed by treatment for 8 hours with 1 mM spermidine. The intracellular concentrations of putrescine, spermidine, and spermine were measured as described above for cells grown under each of these conditions. The addition of DFMO to the growth media resulted in decreased concentrations of intracellular putrescine, spermidine, and spermine compared to control cells, as depicted in FIG. 6. Spermidine concentrations were most affected, showing a nearly 98% decrease in concentration. The concentrations of putrescine and spermine dropped by 60% and 41% respectively. The addition of spermidine (1 mM) to DFMO treated or untreated cells increased spermidine levels by 1.5- and 1.7-fold, respectively, over control cells and restored putrescine and spermine levels to nearly that of control cells. This demonstrates the effectiveness of the using an ODC inhibitor to deplete polyamine levels in examining intracellular polyamine levels.

### EXAMPLE 4

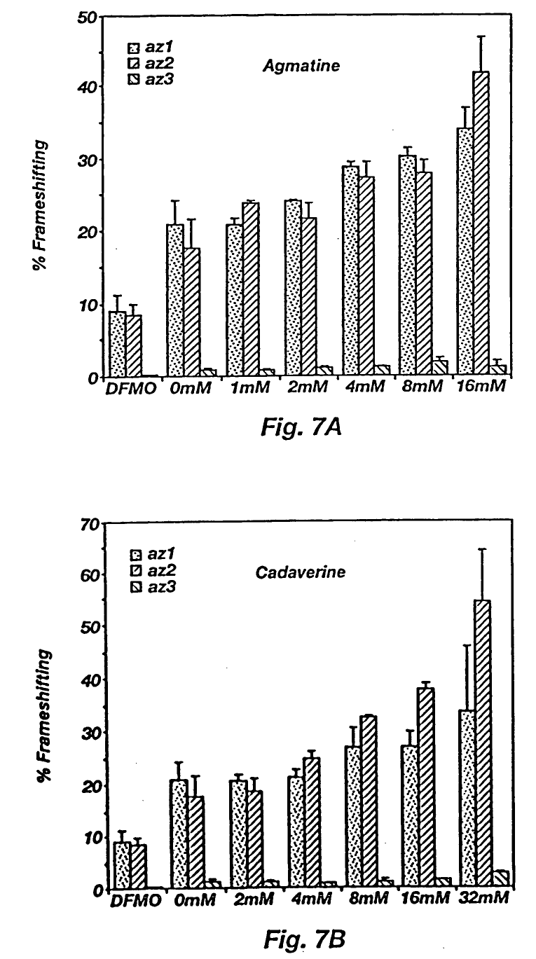

In this example, the procedure of Example 1 was carried out except that the amount of frameshifting was determined in response to the addition of 0-16 mM agmatine to cultured mammalian cells. Agmatine is a polyamine or polyamine analog that does not naturally occur in mammalian cells. Cells treated with 2.5 mM DFMO were used as controls. The plasmids used for transfecting the cells were p2Lucaz1, p2Lucaz2, or p2Luc into which the frameshifting sequence of the antizyme 3 gene was inserted. FIG. 7A shows that in cells transfected with p2Lucaz1 or p2Lucaz2 the addition of exogenous agmatine significantly increased the amount of frameshifting observed. In cells transfected with p2Luc containing the antizyme 3 frameshifting sequence, however, no increase in frameshifting was observed. Therefore, these experiments show that the cultured cell system of the present invention can be used for identifying a small molecule that affects polyamine regulation in such cells.

### EXAMPLE 5

In this example, the procedure of Example 4 was carried out except that the small molecule tested was cadaverine. Cadaverine was added to the cells at levels of 0-32 mM. FIG. 7B shows that cadaverine was effective for significantly increasing the amount of frameshifting observed in mammalian cell transfected with p2Lucaz1 or p2 Lucaz2. In cells transfected with p2Luc containing the antizyme 3 frameshifting sequence, however, no increase in frameshifting was observed. Therefore, these experiments show that the cultured cell system of the present invention can be used for identifying a small molecule that affects polyamine regulation in such cells.

In accordance with the features and combinations described above, an illustrative method of measuring drug induced recoding in the regulation of genes involved in regulating cellular polyamine levels includes:

(a) transfecting a first set of tissue culture cells with a first dual reporter assay construct having two expressible genes separated by a cloning site containing an antizyme frameshift sequence, the first construct containing the downstream gene in a +1 reading frame relative to the upstream gene;

(b) transfecting a second set of tissue culture cells with a second dual reporter assay construct having two expressible genes separated by a cloning site containing an antizyme frameshift sequence, the second construct containing the downstream gene in a 0 reading frame relative to the upstream gene;

(c) growing the transfected cells in conditions that may affect intracellular polyamine levels; and

(d) comparing the ratio of the expression of the two expressible genes in the first set to the ratio of the expression of the two expressible genes in the second set.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been shown in the drawings and fully described above with particularity and detail in connection with what is presently deemed to be the most practical and illustrative embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

The subject matter claimed is:

1. The plasmid p2Lucaz1.

2. The plasmid p2Lucaz2.

3. The plasmid p2Luca1usdel.

4. The plasmid p2Lucaz2usdel.

5. The plasmid p2Lucaz1pkdel.

6. The plasmid p2Lucaz2pkdel.

7. A plasmid for use in assaying cellular polyamine levels comprising:
(a) an upstream DNA segment comprising a first open reading frame encoding a first reporter;
(b) a downstream DNA segment comprising a second open reading frame encoding a second reporter;
(c) a promoter positioned and operative for promoting transcription of the upstream and downstream DNA segments; and
(d) a polyamine-regulated frameshifting sequence positioned between the upstream DNA segment and the downstream DNA segment such that said second open reading frame is in a +1 reading frame with respect to said first open reading frame;
wherein, after transfection of the plasmid into cultured mammalian cells, an effective amount of polyamine stimulates +1 translational frameshifting, thereby resulting in increased expression of the second reporter as compared to expression of the first reporter.

8. The plasmid of claim 7 wherein said first reporter comprises renilla luciferase and said second reporter comprises firefly luciferase.

9. The plasmid of claim 7 wherein said first reporter comprises firefly luciferase and said second reporter comprises renilla luciferase.

10. The plasmid of claim 7 wherein said promoter comprises a promoter functional in mammalian cells.

11. The plasmid of claim 10 wherein said promoter is an SV40 promoter.

12. The plasmid of claim 10 wherein said promoter is a cytomegalovirus promoter.

13. The plasmid of claim 10 wherein said promoter is a eukaryotic polymerase II promoter.

14. The plasmid of claim 7 wherein said polyamine-regulated frameshifting sequence comprises an antizyme 1 polyamine-regulated frameshifting sequence.

15. The plasmid of claim 14 wherein said antizyme 1 polyamine-regulated frameshifting sequence is SEQ ID NO:1.

16. The plasmid of claim 14 wherein said antizyme 1 polyamine-regulated frameshifting sequence is SEQ ID NO:3.

17. The plasmid of claim 14 wherein said antizyme 1 polyamine-regulated frameshifting sequence is SEQ ID NO:5.

18. The plasmid of claim 7 wherein said polyamine-regulated frameshifting sequence comprises an antizyme 2 polyamine-regulated frameshifting sequence.

19. The plasmid of claim 18 wherein said antizyme 2 polyamine-regulated frameshifting sequence is SEQ ID NO:2.

20. The plasmid of claim 18 wherein said antizyme 2 polyamine-regulated frameshifting sequence is SEQ ID NO:4.

21. The plasmid of claim 18 wherein said antizyme 2 polyamine-regulated frameshifting sequence is SEQ ID NO:6.

22. The plasmid of claim 7 wherein said polyamine-regulated frameshifting sequence is an antizyme 3 polyamine-regulated frameshifting sequence.

23. The plasmid of claim 7 wherein said cultured mammalian cells comprise human cells.

24. A plasmid for use in assaying cellular polyamine levels comprising:
(a) an upstream DNA segment comprising a first open reading frame encoding renilla luciferase;
(b) a downstream DNA segment comprising a second open reading frame encoding firefly luciferase;
(c) an SV40 promoter positioned and operative for promoting transcription of the upstream and downstream DNA segments; and
(d) an antizyme polyamine-regulated frameshifting sequence positioned between the upstream DNA segment and the downstream DNA segment such that said second open reading frame is in a +1 reading frame with respect to said first open reading frame;
wherein, after transfection of said plasmid into cultured mammalian cells, an effective amount of polyamine stimulates +1 translation frameshifting at the antizyme polyamine-regulated frameshifting sequence, thereby resulting in increased expression of the firefly luciferase as compared to expression of the renilla luciferase.

25. The plasmid of claim 24 wherein said antizyme polyamine-regulated frameshifting sequence is SEQ ID NO:1.

26. The plasmid of claim 24 wherein said antizyme polyamine-regulated frameshifting sequence is SEQ ID NO:2.

27. The plasmid of claim 24 wherein said antizyme polyamine-regulated frameshifting sequence is SEQ ID NO:3.

28. The plasmid of claim 24 wherein said antizyme polyamine-regulated frameshifting sequence is SEQ ID NO:4.

29. The plasmid of claim 24 wherein said antizyme polyamine-regulated frameshifting sequence is SEQ ID NO:5.

30. The plasmid of claim 24 wherein said antizyme polyamine-regulated frameshifting sequence is SEQ ID NO:6.

31. A plasmid for use in assaying cellular polyamine levels comprising:
(a) an upstream DNA segment comprising a first open reading frame encoding a first reporter;
(b) a downstream DNA segment comprising a second open reading frame encoding a second reporter;
(c) a promoter positioned and operative for promoting transcription of the upstream and downstream DNA segments; and
(d) a polyamine-regulated frameshifting sequence positioned between the upstream DNA segment and the downstream DNA segment such that said second open reading frame is in a different reading frame with respect to said first open reading frame;
wherein, after transfection of the plasmid into cultured mammalian cells, an effective amount of polyamine stimulates translational frameshifting such that the first open reading frame and the second open reading frame are translated in the same frame, thereby resulting in increased expression of the second reporter as compared to expression of the first reporter.

32. A method of estimating recoding in genes involved in regulating cellular polyamine levels comprising:
(a) transfecting a first set of cultured mammalian cells with a first dual reporter assay construct comprising
(i) an upstream DNA segment comprising a first open reading frame encoding a first reporter.
(ii) a downstream DNA segment comprising a second open reading frame encoding a second reporter,
(iii) a promoter positioned and operative for promoting transcription of the upstream and downstream DNA segments, and
(iv) a polyamine-regulated frameshifting sequence positioned between the upstream DNA segment and the downstream DNA segment such that said second open reading frame is in a +1 reading frame with respect to said first open reading frame;
(b) transfecting a second set of cultured mammalian cells with a second dual reporter assay construct comprising said first dual reporter assay construct except that said second open reading frame is in the same reading frame with respect to said first open reading frame;
(c) growing the transfected first set of cultured mammalian cells and the transfected second set of cultured mammalian cells and determining a ratio of levels of expression of the second reporter compared to the first reporter in each of the first set and the second set of cultured mammalian cells; and
(d) comparing each said ratio, wherein a proportion of the ratio for the first set of cultured mammalian cells to the ratio for the second set of cultured mammalian cells is an estimate of recoding in the genes involved in regulating cellular polyamine levels.

33. The method of claim 32 wherein said first reporter comprises renilla luciferase and said second reporter comprises firefly luciferase.

34. The method of claim 32 wherein said first reporter comprises firefly luciferase and said second reporter comprises renilla luciferase.

35. The method of claim 32 wherein said promoter comprises a promoter functional in mammalian cells.

36. The method of claim 35 wherein said promoter is an SV40 promoter.

37. The method of claim 35 wherein said promoter is a cytomegalovirus promoter.

38. The method of claim 35 wherein said promoter is a eukaryotic polymerase II promoter.

39. The method of claim 32 wherein said polyamine-regulated frameshifting sequence comprises an antizyme 1 polyamine-regulated frameshifting sequence.

40. The method of claim 39 wherein said antizyme 1 polyamine-regulated frameshifting sequence is SEQ ID NO:1.

41. The method of claim 39 wherein said antizyme 1 polyamine-regulated frameshifting sequence is SEQ ID NO:3.

42. The method of claim 39 wherein said antizyme 1 polyamine-regulated frameshifting sequence is SEQ ID NO:5.

43. The method of claim 32 wherein said polyamine-regulated frameshifting sequence comprises an antizyme 2 polyamine-regulated frameshifting sequence.

44. The method of claim 43 wherein said antizyme 2 polyamine-regulated frameshifting sequence is SEQ ID NO:2.

45. The method of claim 43 wherein said antizyme 2 polyamine-regulated frameshifting sequence is SEQ ID NO:4.

46. The method of claim 43 wherein said antizyme 2 polyamine-regulated frameshifting sequence is SEQ ID NO:6.

47. The method of claim 32 wherein said polyamine-regulated frameshifting sequence is an antizyme 3 polyamine-regulated frameshifting sequence.

48. The method of claim 32 wherein said cultured mammalian cells comprise human cells.

49. The method of claim 32 further comprising treating the cultured mammalian cells such that endogenous levels of polyamines are reduced.

50. The method of claim 49 wherein said treating the cultured mammalian cells such that endogenous levels of polyamines are reduced comprises treating the cultured mammalian cells with an inhibitor of polyamine biosynthesis.

51. The method of claim 50 wherein said inhibitor of polyamine biosynthesis comprises an inhibitor of ornithine decarboxylase.

52. The method of claim 51 wherein the inhibitor of ornithine decarboxylase is difluoromethylornithine.

53. The method of claim 50 wherein said inhibitor of polyamine biosynthesis comprises an inhibitor of S-adenosyl methionine decarboxylase.

54. The method of claim 49 wherein said treating the cultured mammalian cells such that endogenous levels of polyamines are reduced comprises treating the cultured mammalian cells with an stimulator of polyamine excretion or catabolism.

55. The method of claim 54 wherein said stimulator of polyamine excretion or metabolism stimulates spermidine/spermine N′-acetyltransferase (SSAT) activity.

56. A method for screening for small molecules that affect polyamine regulation in cells, comprising:
(a) transfecting a first set of cultured mammalian cells with a first dual reporter assay construct comprising
(i) an upstream DNA segment comprising a first open reading frame encoding a first reporter,
(ii) a downstream DNA segment comprising a second open reading frame encoding a second reporter,
(iii) a promoter positioned and operative for promoting transcription of the upstream and downstream DNA segments, and
(iv) a polyamine-regulated frameshifting sequence positioned between the upstream DNA segment and the downstream DNA segment such that said second open reading frame is in a +1 reading frame with respect to said first open reading frame;
(b) transfecting a second set of cultured mammalian cells with a second dual reporter assay construct comprising said first dual reporter assay construct except that said second open reading frame is in the same reading frame with respect to said first open reading frame;
(c) growing the first set of cultured mammalian cells and the second set of cultured mammalian cells in the presence of a candidate small molecule and determining a ratio of levels of expression of the second reporter compared to the first reporter for each of the first set and the second set of cultured mammalian cells; and
(d) comparing each said ratio, wherein an increase or decrease in the ratio indicates that the small molecule affects polyamine regulation.

57. The method of claim 56 wherein said first reporter comprises renilla luciferase and said second reporter comprises firefly luciferase.

58. The method of claim 56 wherein said first reporter comprises firefly luciferase and said second reporter comprises renilla luciferase.

59. The method of claim 56 wherein said promoter comprises a promoter functional in mammalian cells.

60. The method of claim 59 wherein said promoter is an SV40 promoter.

61. The method of claim 59 wherein said promoter is a cytomegalovirus promoter.

62. The method of claim 59 wherein said promoter is a eukaryotic polymerase II promoter.

63. The method of claim 56 wherein said polyamine-regulated frameshifting sequence comprises an antizyme 1 polyamine-regulated frameshifting sequence.

64. The method of claim 63 wherein said antizyme 1 polyamine-regulated frameshifting sequence is SEQ ID NO:1.

65. The method of claim 63 wherein said antizyme 1 polyamine-regulated frameshifting sequence is SEQ ID NO:3.

66. The method of claim 63 wherein said antizyme 1polyamine-regulated frameshifting sequence is SEQ ID NO:5.

67. The method of claim 56 wherein said polyamine-regulated frameshifting sequence comprises an antizyme 2 polyamine-regulated frameshifting sequence.

68. The method of claim 67 wherein said antizyme 2 polyamine-regulated frameshifting sequence is SEQ ID NO:2.

69. The method of claim 67 wherein said antizyme 2 polyamine-regulated frameshifting sequence is SEQ ID NO:4.

70. The method of claim 67 where in said antizyme 2 polyamine-regulated frameshifting sequence is SEQ ID NO:6.

71. The method of claim 56 wherein said polyamine-regulated frameshifting sequence is an antizyme 3 polyamine-regulated frameshifting sequence.

72. The method of claim 56 wherein said cultured mammalian cells comprise human cells.

73. The method of claim 56 further comprising treating the first set of cultured mammalian cells and the second set of cultured mammalian cells such that endogenous levels of polyamnines are reduced.

74. The method of claim 73 wherein said treating the first set of cultured mammalian cells and the second set of cultured mammalian cells such that endogenous levels of polyamines are reduced comprises treating the cultured mammalian cells with an inhibitor of polyamine biosynthesis.

75. The method of claim 74 wherein said inhibitor of polyamine biosynthesis comprises an inhibitor of ornithine decarboxylase.

76. The method of claim 75 wherein the inhibitor of ornithine decarboxylase is difluoromethylornithine.

77. The method of claim 74 wherein said inhibitor of polyamine biosynthesis comprises an inhibitor of S-adenosyl methionine decarboxylase.

78. The method of claim 73 wherein said treating the first set of cultured mammalian cells and the second set of cultured mammalian cells such that endogenous levels of polyamines are reduced comprises treating the cultured mammalian cells with an stimulator of polyamine excretion or catabolism.

79. The method of claim 78 wherein said stimulator of polyamine excretion or metabolism comprises spermidine/spermine N′-acetyltransferase (SSAT).

80. A method for screening for small molecules that affect polyamine regulation in cells, comprising:
(a) transfecting a first set of cultured mammalian cells with a first dual reporter assay construct comprising
(i) an upstream DNA segment comprising a first open reading frame encoding a first reporter,
(ii) a downstream DNA segment comprising a second open reading frame encoding a second reporter,
(iii) a promoter positioned and operative for promoting transcription of the upstream and downstream DNA segments, and
(iv) a polyamine-regulated frameshifting sequence positioned between the upstream DNA segment and the downstream DNA segment such that said second open reading frame is in a +1 reading frame with respect to said first open reading frame;
(b) transfecting a second set of cultured mammalian cells with a second dual reporter assay construct comprising said first dual reporter assay construct except that said second open reading frame is in the same reading frame with respect to said first open reading frame;
(c) treating the first set of cultured mammalian cells and the second set of cultured mammalian cells such that endogenous levels of polyamines are reduced;
(d) growing the first set of cultured mammalian cells and the second set of cultured mammalian cells in the presence of a candidate small molecule and determining a ratio of levels of expression of the second reporter compared to the first reporter for each of the first set and the second set of cultured mammalian cells; and
(e) comparing each said ratio, wherein an increase or decrease in the ratio indicates that the small molecule affects polyamine regulation.

81. The method of claim 80 wherein said first reporter comprises renilla luciferase and said second reporter comprises firefly luciferase.

82. The method of claim 80 wherein said first reporter comprises firefly luciferase and said second reporter renilla luciferase.

83. The method of claim 80 wherein said promoter comprises a promoter functional in mammalian cells.

84. The method of claim 83 wherein said promoter is an SV40 promoter.

85. The method of claim 83 wherein said promoter is a cytomegalovirus promoter.

86. The method of claim 83 wherein said promoter is a eukaryotic polymerase II promoter.

87. The method of claim 80 wherein said polyamine-regulated frameshifting sequence comprises an antizyme 1 polyamine-regulated frameshifting sequence.

88. The method of claim 87 wherein said antizyme 1 polyamine-regulated frameshifting sequence is SEQ ID NO:1.

89. The method of claim 87 wherein said antizyme 1 polyamine-regulated frameshifting sequence is SEQ ID NO:3.

90. The method of claim 87 wherein said antizyme 1 polyamine-regulated frameshifting sequence is SEQ ID NO:5.

91. The method of claim 80 wherein said polyamine-regulated frameshifting sequence comprises an antizyme 2 polyamine-regulated frameshifting sequence.

92. The method of claim 91 wherein said antizyme 2 polyamine-regulated frameshifting sequence is SEQ ID NO:2.

93. The method of claim 91 wherein said antizyme 2 polyamine-regulated frameshifting sequence is SEQ ID NO:4.

94. The method of claim 91 where in said antizyme 2 polyamine-regulated frameshifting sequence is SEQ ID NO:6.

95. The method of claim 80 wherein said polyamine-regulated frameshifting sequence is an antizyme 3 polyamine-regulated frameshifting sequence.

96. The method of claim 80 wherein said cultured mammalian cells comprise human cells.

97. The method of claim 80 wherein said treating the first set of cultured mammalian cells and the second set of cultured mammalian cells such that endogenous levls of polyamines are reduced comprises treating the cultred mammalian cells with an inhbitor of polyamine biosynthesis.

98. The method of of claim 97 wherein said inhbitor of polyamine biosynthesis comprises an inhibitor of ornithine decarboxylase.

99. The method of claim 98 wherein inhibitor of ornithine decarboxylase is difluoromethylornithine.

100. The method of claim 97 wherein said inhibitor of polyamine biosynthesis comprises an inhibitor of S-adenosyl methionine decarboxylase.

101. The method of claim 80 wherein said treating the first set of cultured mammalian cells and the second set of cultured mammalian cells such that endogenous levels of polyamines are reduced comprises treating the cultured mammalian cells with an stimulator of polyamine excretion or catabolism.

102. The method of claim 101 wherein said stimulator of polyamine excretion or metabolism comprises spermidine/spermine N′-acetyltransferase (SSAT).

103. A method for screening for small molecules that affect translational frameshifting in cells, comprising:
(a) transfecting a first set of cultured mammalian cells with a first dual reporter assay construct comprising
(i) an upstream DNA segment comprising a first open reading frame encoding a first reporter,
(ii) a downstream DNA segment comprising a second open reading frame encoding a second reporter,
(iii) a promoter positioned and operative for promoting transcription of the upstream and downstream DNA segments, and
(iv) a polyamine-regulated frameshifting sequence positioned between the upstream DNA segment and the downstream DNA segment such that said second open reading frame is in a different reading frame with respect to said first open reading frame;
(b) transfecting a second set of cultured mammalian cells with a second dual reporter assay construct comprising said first dual reporter assay construct except that said second open reading frame is in the same reading frame with respect to said first open reading frame;
(c) growing the first set of cultured mammalian cells and the second set of cultured mammalian cells in the presence of a candidate small molecule and determining a ratio of levels of expression of the second reporter compared to the first reporter for each of the first set and the second set of cultured mammalian cell; and
(d) comparing each said ratio wherein an increase or decrease in the ratio indicates that the small molecule affects translational frameshifting.

104. The method of claim 103 wherein said first reporter comprises renilla luciferase and said second reporter comprises firefly luciferase.

105. The method of claim 103 wherein said first reporter comprises firefly luciferase and said second reporter comprises renilla luciferase.

106. The method of claim 103 wherein said promoter comprises a promoter functional in mammalian cells.

107. The method of claim 106 wherein said promoter is an SV40 promoter.

108. The method of claim 106 wherein said promoter is a cytomegalovirus promoter.

109. The method of claim 106 wherein said promoter is a eukaryotic polymerase II promoter.

110. The method of claim 103 wherein said polyamine-regulated frameshifting sequence comprises an antizyme 1 polyamine-regulated frameshifting sequence.

111. The method of claim 110 wherein said antizyme 1 polyamine-regulated frameshifting sequence is SEQ ID NO:1.

112. The method of claim 110 wherein said antizyme 1 polyamine-regulated frameshifting sequence is SEQ ID NO:3.

113. The method of claim 110 wherein said antizyme 1polyamine-regulated frameshifting sequence is SEQ ID NO:5.

114. The method of claim 103 wherein said polyamine-regulated frameshifting sequence comprises an antizyme 2 polyamine-regulated frameshifting sequence.

115. The method of claim 114 wherein said antizyme 2 polyamine-regulated frameshifting sequence is SEQ ID NO:2.

116. The method of claim 114 wherein said antizyme 2 polyamine-regulated frameshifting sequence is SEQ ID NO:4.

117. The method of claim 114 where in said antizyme 2 polyamine-regulated frameshifting sequence is SEQ ID NO:6.

118. The method of claim 103 wherein said polyamine-regulated frameshifting sequence is an antzyme 3 polyamine-regulated frameshifting sequence.

119. The method of claim 103 wherein said cultured mammalian cells comprise human cells.

120. The method of claim 103 further treating the first set of cultured mammalian cells and the second set of cultured mammalian cells such that endogenous levels of polyamines are reduced.

121. The method of claim 120 wherein said treating the first set of cultured mammalian cells and the second set of cultured mammalian cells such that endogenous levels of polyamines are reduced comprises treating the cultured mammalian cells with an inhibitor of polyamine biosynthesis.

122. The method of claim 121 wherein said inhibitor of polyamine biosynthesis comprises an inhibitor of ornithine decarboxylase.

123. The method of claim 122 wherein the inhibitor of ornithine decarboxylase is difluoromethylornithine.

124. The method of claim 121 wherein said inhibitor of polyamine biosynthesis comprises an inhibitor of S-adenosyl methionine decarboxylase.

125. The method of claim 120 wherein said treating the first set of cultured mammalian cells and the second set of cultured mammalian cells such that endogenous levels of polyamines are reduced comprises treating the cultured mammalian cells with an stimulator of polyamine excretion or catabolism.

126. The method of claim 125 wherein said stimulator of polyamine excretion or metabolism comprises spermidine/spermine N′-acetyltransferase (SSAT).

127. A method for screening for small molecules that affect translational frameshifting in cells, comprising:
(a) transfecting a first set of cultured mammalian cells with a first dual reporter assay construct comprising
(i) an upstream DNA segment comprising a first open reading frame encoding a first reporter,
(ii) a downstream DNA segment comprising a second open reading frame encoding a second reporter,
(iii) a promoter positioned and operative for promoting transcription of the upstream and downstream DNA segments, and
(iv) a polyamine-regulated frameshifting sequence positioned between the upstream DNA segment and the downstream DNA segment such that said second open reading frame is in a different reading frame with respect to said first open reading frame;
(b) transfecting a second set of cultured mammalian cells with a second dual reporter assay construct comprising said first dual reporter assay construct except that said second open reading frame is in the same reading frame with respect to said first open reading frame;
(c) treating the first set of cultured mammalian cells and the second set of cultured mammalian cells such that endogenous levels of polyamines are reduced;
(d) growing the first set of cultured mammalian cells and the second set of cultured mammalian cells in the presence of a candidate small molecule and determining a ratio of levels of expression of the second reporter compared to the first reporter for each of the first set and the second set of cultured mammalian cells; and
(e) comparing each said ratio, wherein an increase or decrease in the ratio indicates that the small molecule affects translational frameshifting.

128. The method of claim 127 wherein said first reporter comprises renilla luciferase and said second reporter comprises firefly luciferase.

129. The method of claim 127 wherein said first reporter comprises firefly luciferase and said second reporter comprises renilla luciferase.

130. The method of claim 127 wherein said promoter comprises a promoter functional in mammalian cells.

131. The method of claim 130 wherein said promoter is an SV40 promoter.

132. The method of claim 130 wherein said promoter is a cytomegalovirus promoter.

133. The method of claim 130 wherein said promoter is a eukaryotic polymerase II promoter.

134. The method of claim 127 wherein said polyamine-regulated frameshifting sequence comprises an antizyme 1 polyamine-regulated frameshifting sequence.

135. The method of claim 134 wherein said antizyme 1 polyamine-regulated frameshifting sequence is SEQ ID NO:1.

136. The method of claim 134 wherein said antizyme 1 polyamine-regulated frameshifting sequence is SEQ ID NO:3.

137. The method of claim 134 wherein said antizyme 1polyamine-regulated frameshifting sequence is SEQ ID NO:5.

138. The nethod of claim 127 wherein said polyamine-regulated frameshifting sequence comprises an antizyme 2 polyamine-regulaled frameshifting sequence.

139. The method of claim 138 wherein said antizyme 2 polyamine-regulated frameshifting sequence is SEQ ID NO:2.

140. The method of claim 138 wherein said antizyme 2 polyamine-regulated frameshiftmg sequence is SEQ ID NO:4.

141. The method of claim 138 wherein said antizyme 2 polyamine-regulated frameshifting sequence is SEQ ID NO:6.

142. The method of claim 127 wherein said polyamine-regulated frameshifting sequence is an antizyme 3 polyamine-regulated frameshifting sequence.

143. The method of claim 127 wherein said cultured mammalian cells comprise human cells.

144. The method of claim 127 wherein said treating the first set of cultured mammalian cells and the second set of cultured mammalian cells such that endogenous levels of polyamines are reduced comprises treating the cultured mammalian cells with an inhibitor of polyamine biosynthesis.

145. The method of claim 144 wherein said inhibitor of polyamine biosynthesis comprises an inhibitor of ornithine decarboxylase.

146. The method of claim 145 wherein the inhibitor of ornithine decarboxylase is difluoromethylornithine.

147. The method of claim 144 wherein said inhibitor of polyanmine biosynthesis comprises an inhibitor of S-adenosyl methionine decarboxylase.

148. The method of claim 127 wherein said treating the first set of cultured mammalian cells and the second set of cultured mammalian cells such that endogenous levels of polyamines are reduced comprises treating the cultured mammalian cells with an stimulator of polyamine excretion or catabolism.

149. The method of claim 148 wherein said stimulator of polyamine excretion or metabolism comprises spermidine/spermine N′-acetyltransferase (SSAT).
